(19) United States
(12) Patent Application Publication
(10) Pub. No.: US 2005/0177107 A1
(43) Pub. Date: Aug. 11, 2005

(54) Safety syringe assembly

(76) Inventors: Peter Balfour Dugmore, Cape Town (ZA); Anthony Eric Bull, Cape Town (ZA); Stanford William Gladwin Reynolds, Westville (ZA)

Correspondence Address: DLA PIPER RUDNICK GRAY CARY US LLP, P. O. BOX 64807, CHICAGO, IL, 60664-0807, US

(21) Appl. No.: 10/275,937

(22) PCT Filed: May 10, 2001
(86) PCT No.: PCT/IB01/00880

(30) Foreign Application Priority Data
May 10, 2000 (ZA) 2000/2283

(51) Int. Cl. A61M 5/00

(52) U.S. Cl. 604/110

(57) ABSTRACT

A hypodermic safety syringe assembly (10) includes a cylindrical barrel (12) having a front end defining a first aperature (14) and a rear end defining a second aperature (16), a closure member (32) shaped to engage the barrel (12) at or adjacent the front end thereof to seal the first aperature (14), and a piston assembly (18) reciprocally movable within the barrel (12) and includes a retractor (20) terminating in a piston formation (24) engageable with the closure member (32) to disengage the closure member from the front end of the barrel so that a hypodermic needle (40) attached thereto can be withdrawn into the barrel (12) after use, wherein the closure member (32) includes a barrel engaging formation (44,48) releasably engageable within a closure member engaging formation (64) at the front end of the barrel (12), and a piston coupling formation (50,54) for engaging a closure member coupling formation (68,69) on the piston (24), with inter-engagement of the piston and closure member coupling formations (50,54;68,69) being arranged to facilitate disengagement of the barrel engaging and closure member engaging formations (64; 44,48) from one another and to allow withdrawal of the piston assembly (20,24), closure member (32) and hypodermic needle (40) into the barrel (12).

### BACKGROUND OF THE INVENTION

This invention relates to a hypodermic safety syringe assembly.

The needles of used hypodermic syringes pose an increasing threat of the transmissibility of potentially lethal infections such as HIV⁺ and hepatitis B viruses, not only to persons administering the injection, but also to persons who are charged with handling such equipment immediately after use, to others who are exposed to the possibility of accidental, post-injection contact in environments in which hypodermic procedures are routinely administered, and to still others who might come into contact with used syringes which have been inadequately disposed of.

Various safety hypodermic syringes have been designed which attempt to minimise these risks. These include various forms of needle containment and needle disposal units which respectively seek to encapsulate a used needle or allow such used needle to be destroyed by incineration or the like. In order to be effective, needle destroying devices have to be available in situ at all locations where such procedures are undertaken. The effectiveness of needle destroying devices is further reduced by the fact that the sharp end of the used needle may typically be exposed for an appreciable period of time before it is destroyed.

Another arrangement which is available comprises a cylindrical sleeve which fits around the barrel of a syringe, and which can be extended forward over the needle attached to the syringe after use. When the sleeve is fully extended, locking formations on the sleeve engage complemental locking formations formed on or adjacent the end of the syringe barrel so that the sleeve cannot be retracted.

Drawbacks of protective sleeve arrangements include the fact that in a number of such devices the shield has an open end, which is large enough to allow a user's finger to enter and to contact the needle. A directly concomitant disadvantage is the fact that in order to obviate this danger, the needle used has to be substantially shorter than the length of the syringe barrel and shield. The extended and locked syringe/sleeve assembly is relatively long and bulky, and uses a substantial additional mass of plastic material compared with the syringe alone. Arrangements of this type are also relatively complicated to manufacture, frequently involving a complete re-engineering of the syringe in order for it to co-operate in secure locking engagement with the sleeve. Examples are provided by the disclosures in U.S. Pat. Nos. 5,053,018 and 5,304,149, both of which teach a hypodermic syringe and needle assembly on which is mounted a shield which is movable between a retracted position in which the needle point is exposed and an extended position in which the needle point is covered.

User resistance has been encountered to the added bulk and overall diameter of the assembly contributed by the sleeve, which is particularly disadvantageous when the needle point needs to be acutely tangential to the surface of the skin of the patient. The fact that the contents and the graduations on the syringe must be viewed through the material of the shield has also proved inconvenient and can lead to errors in use.

An alternative arrangement where the syringe is substantially re-engineered typically proposes a device having a barrel, sheath and piston, the piston being provided with a liquid passageway interconnectable with the barrel. The injection of fluid is accomplished by moving the barrel relative to the sheath (as opposed to the more familiar approach of moving the piston relative to the barrel, in conventional hypodermic syringe assemblies). An example of this approach is provided by South African Patent 93/5302, which teaches the automatic and non-discretionary single use safe rendition of a needle.

Three disadvantages of this approach have manifested themselves in practice. The first is the fact that the procedure represents a significant departure from the operation of a conventional syringe. Nursing staff have shown resistance to the required adjustment from what is a comfortable and familiar routine. A further disadvantage is the fact that this approach requires a large number of component parts, which adds significantly to the cost of both the moulds and mould operations required and of the materials used. A final disadvantage is the impression left with the practitioner that the precise point when the device is rendered needle-safe is no longer a matter of professional choice, but is rather a matter of when the manufacturer has prescribed that it is advisable or necessary.

The highest incidence of the HIV⁺ and hepatitis viruses generally occur in poor, third world countries which can ill afford devices reliant on significant re-engineering, which do not take into account the high level of the capital invested world-wide in manufacturing facilities for and stocks of conventional hypodermic syringes and needles.

It is an object of the invention to provide an alternative safety syringe assembly which allows for an administering procedure which does not depart significantly from that of a conventional syringe, and which does not utilise an additional outer sheath or shield.

### SUMMARY OF THE INVENTION

According to a first aspect of the invention there is provided a hypodermic safety syringe assembly including a cylindrical barrel having a front end defining a first aperture and a rear end defining a second aperture, a closure member shaped to engage the barrel at or adjacent the front end thereof to seal the first aperture, and a piston assembly reciprocally movable within the barrel and including a retractor terminating in a piston formation engageable with the closure member to disengage the closure member from the front end of the barrel so that a hypodermic needle attached thereto can be withdrawn into the barrel after use, wherein the closure member includes a barrel engaging formation releasably engageable within a closure member engaging formation at the front end of the barrel, and a piston coupling formation for engaging a closure member coupling formation on the piston, with inter-engagement of the piston and closure member coupling formations being arranged to facilitate disengagement of the barrel engaging and closure member engaging formations from one another and to allow withdrawal of the piston assembly, closure member and hypodermic needle into the barrel.

The closure member preferably includes an inner skirt portion connected to an outer skirt portion by a hinge, with a displacement groove located between the inner and outer skirt portions.

The barrel engaging formation preferably includes a lip extending from the outer skirt portion, and the closure member engaging formation preferably includes a lip receiving recess adjacent the front end of the barrel.

The piston coupling formation preferably includes at least one first projection and a corresponding projection receiving recess located radially outwardly of the first projection, and the closure member coupling formation preferably includes a second projection and a corresponding recess located radially inwardly of the second projection, with the second projection being arranged to displace the first projection inwardly about the hinge during inter-engagement of the piston coupling formation and closure member coupling formation so as to urge the outer skirt formation, from which the first projection depends, inwardly so as to disengage the lip from the lip receiving recess.

The first projection is preferably a first annular lobe formation and the second projection is preferably a second annular lobe formation.

The assembly preferably includes detaining means for detaining the used needle in a locked safe position within the barrel.

An embodiment of the invention is described in detail in the following passages of the specification which refer to the accompanying drawings. The drawings, however, are merely illustrative of how the invention might be put into effect, so that the specific form and arrangement of the features shown is not to be understood as limiting on the invention.

### BRIEF DESCRIPTION OF THE DRAWINGS

FIG. 1 shows a partly cross-sectioned side view of a safety syringe assembly according to the invention;

FIG. 2A shows an underplan view of a closure member forming part of the safety syringe assembly of FIG. 1;

FIG. 2B shows a cross-sectional side view along the line 2B-2B of FIG. 2A;

FIGS. 3A to 3C show cross-sectional detailed side views of the manner in which the closure member engages the front end of the barrel during assembly of the safety syringe assembly of the invention;

FIGS. 3D to 3H show, stepwise, the procedure involved in the piston formation engaging with and retracting the closure member into the barrel of the safety syringe assembly;

FIG. 4 shows a partly cross-sectioned side detailed view of the piston formation and closure member in a retracted engaged position; and

FIG. 5 shows a partly cross-sectioned side view of the safety syringe in the retracted engaged position.

### DETAILED DESCRIPTION OF THE DRAWINGS

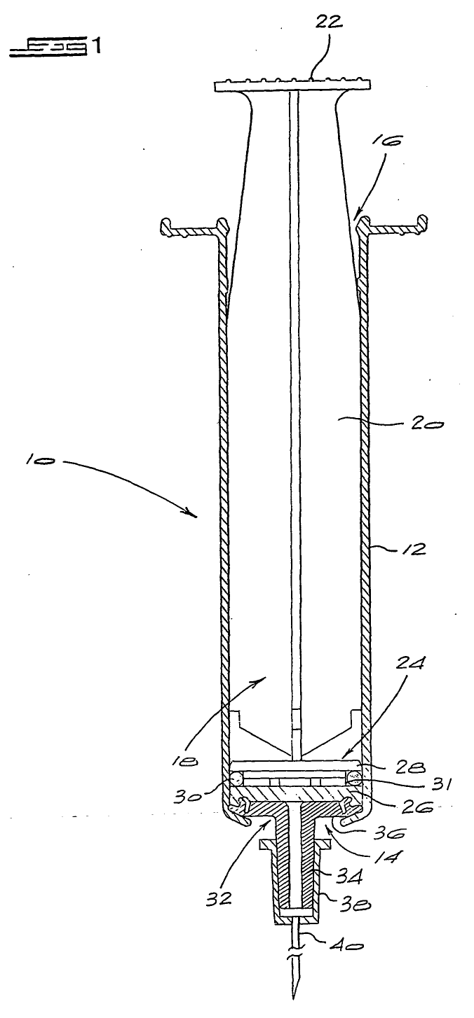

Referring first to FIG. 1, safety syringe assembly 10 of the invention comprises a barrel 12 having a front opening 14 and a rear opening 16. A piston assembly 18 is arranged to travel reciprocally within the barrel 12 and includes an elongate retractor 20 having a cruciform profile terminating in a rear finger disc 22. A piston formation 24 extends from the front end of the retractor, and includes front and rear piston discs 26 and 28 and an O-ring 30 located within an intervening annular recess 31. The front piston disc 26 is coupled to a closure member 32 formed with a central spigot formation 34 extending from a skirt 36. A needle hub 38 fits over the needle spigot 34 in a snug friction fit, and a needle 40 extends from the needle hub 38.

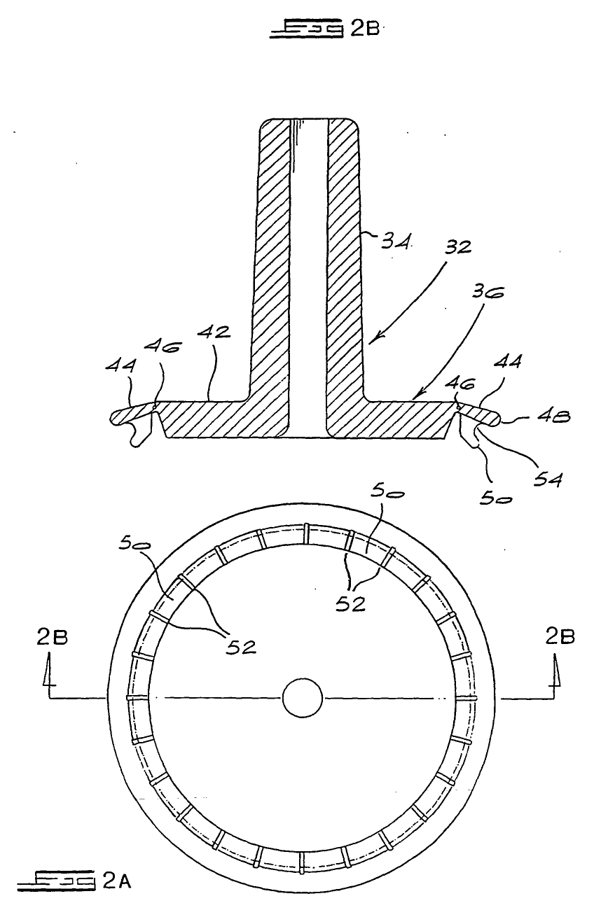

Referring now to FIGS. 2A and 2B, the skirt 36 of the closure member 32 includes a rigid inner skirt portion 42 and an outer peripheral relatively flexible skirt portion 44 which is arranged to flex about a narrowed neck portion defining a radially hooped hinge 46. The outer peripheral flexible skirt portion 44 terminates in a barrel-engaging lip 48. A series of piston-engaging lobes 50 extend axially inwardly and radially outwardly from the back of the flexible skirt portion 44. The lobes 50 are arranged in an annulus, with a total of 24 lobes being defined by evenly spaced gaps 52 therebetween which are designed to reduce hoop stress and facilitate inward flexure of the flexible skirt portion 44. A piston-receiving recess 54 is defined between the free ends of the lobes 50 and the lip 48 of the flexible skirt. There is a displacement groove 74 between the inner skirt portion 42 and outer skirt portion 44.

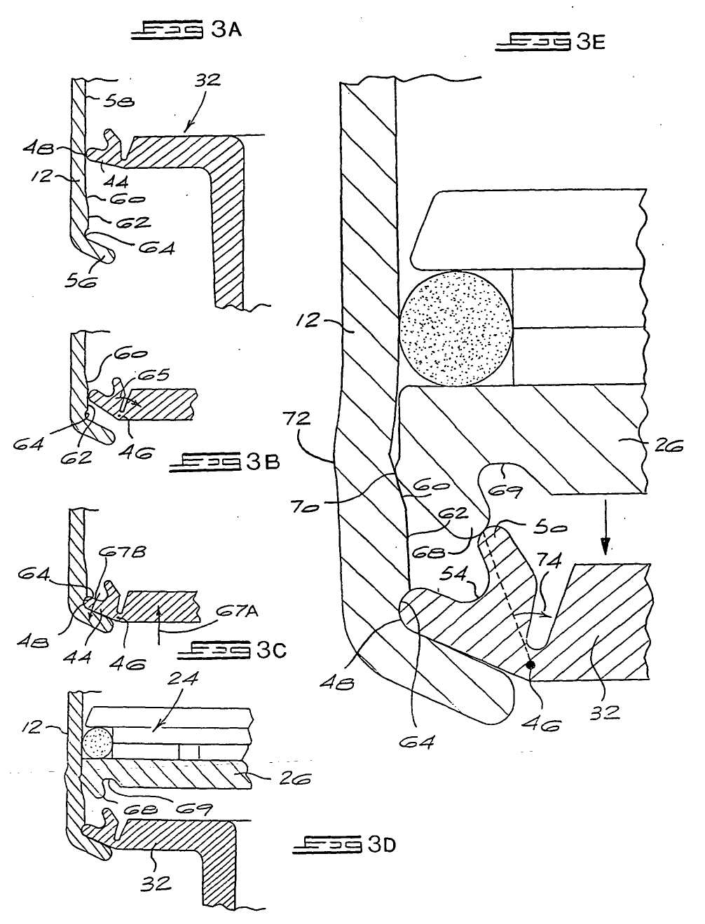

Referring now to FIGS. 3A to 3C, the syringe barrel 12 terminates in an inwardly extending flange 56, and defines a round cylindrical inner surface 58 which extends for almost the entire length thereof. A ramp 60 extends between the cylindrical surface 58 and a reduced diameter land 62, and a lip-locating recess or groove 64 is located between the land 62 and the flange 56. The closure member 32 is fitted to the syringe barrel by inserting it through the rear opening 16 thereof. As the closure member 32 travels down the barrel, the flexible skirt portion 44 is marginally flexed inwardly as the lip 48 contacts the inner surface 58 of the barrel. As the lip 48 rides up the ramp 60 and onto the reduced diameter land 62, the outer flexible skirt portion 44 flexes inwardly in the direction of arrow 65 about the hinge 46, as is clear from FIG. 3B. Further forward movement of the closure member 32 causes the barrel-engaging lip 48 to locate firmly within the lip receiving recess 64 at the front end of the barrel, with the outer skirt portion 44 being in a partly inwardly flexed position.

When the closure member 32 is in the forward engaged position of FIG. 3C, a pushing force in the direction of arrow 67A translates into an outwardly pivoting movement of the outer flexible skirt portion 44 in the direction of arrow 67B. This has the effect of expanding the outer diameter of the skirt portion and jamming the lip 48 of the skirt even more tightly within the detent or recess 64.

After administration of the injection, the piston formation 24 is moved forwards within the barrel 12 to the position indicated in FIG. 3D. The front piston disc 26 is formed with an outer closure member-engaging lobe 68 and an adjacent lobe-receiving recess 69 for receiving the lobe 50 of the closure member 32. As the piston formation 24 advances to the FIG. 3E position, an outer peripheral rib formation 70 bears firmly against the ramp 60. As the outer diameter of the rib 70 is greater than the progressively decreasing inner diameter of the ramp 60, this results in the relatively rigid front piston disc 26 beginning to outwardly deform the syringe barrel 12, as is indicated by bulge 72. The barrel 12 is typically formed from a softer grade of polypropylene than the piston formation to facilitate the outward deformation of the barrel walls.

In the FIG. 3E position, the geometry of the arrangement is such that the outermost points of the first piston-engaging lobes 50 and the second annular closure member-engaging lobe 68 are tangential. Further advancement of the piston formation induces a complex combination of forces which ultimately result in the first set of lobes 50 rotating inwardly about the hinge 46 in the direction of the arrow in the displacement groove 74. The displacement groove 74 permits inward rotation of the first set of lobes 50.

In the engaged position of FIG. 3G, the piston formation 24 and the closure member 32 are fully inter-engaged, with the second annular lobe 68 nesting within the recess 54 defined between the lobe 50 and the outer lip 48, and the first set of lobes 50 nesting within the annular recess 69 defined within the front piston disc 26. In this position, further outward deformation of the barrel occurs at 76. The extent of deformation can clearly be seen in FIG. 3F, where the degree of overlap between the front piston disc 26 and the non-deformed barrel wall 12 is shown at 78. The radially inward movement of the lobe 50 results in the lip 48 being disengaged from the recess 64 and a clearance gap 80 being defined. As is clear from FIG. 3H, the now coupled piston assembly 18 and closure member 32 can be retracted by virtue of the simultaneous inter-engagement of the lobes 50 and 68 and the disengagement of the lip 48 from the recess 64.

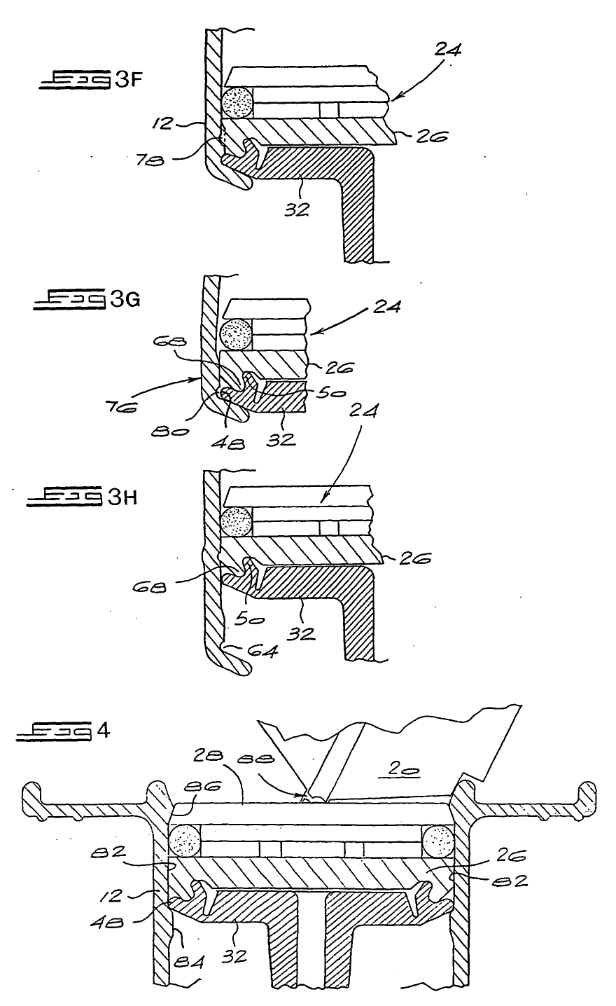

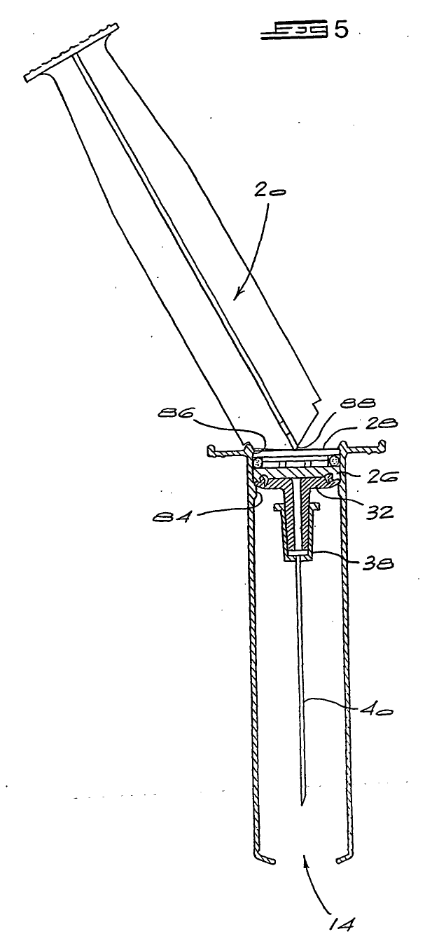

The piston assembly 18, closure member 32 and needle hub assembly including the used needle 40 and the needle hub 38 are subsequently retracted fully to the FIGS. 4 and 5 position in which the used needle point is locked safely within the syringe barrel, with the narrow diameter opening 14 constituting a finger barrier. It can clearly be seen how the inner surface of the barrel wall 12 has been profiled with a piston formation and closure member-detaining land 82 being defined between a front rib or detent 84 and a rear rib or detent 86 of reduced diameter, with the rear rib 86 being sufficiently prominent to prevent both the rear and front piston discs 28 and 26 respectively from being retracted out of the barrel. The lip 48 of the outer skirt portion is located just behind the rib 84 which detains it in the fully retracted position, and prevents subsequent forward movement of the inter-engaged piston formation and closure member which carries the needle hub and used needle 40. A frangible connection 88 is provided between the rear piston disc 28 and the retractor 20. The frangible connection 88 has been designed so that it has both tensile and compressive strength in an axial direction, but is weak in the shear vector which allows the retractor to be broken off in the manner illustrated. Both the retractor and the syringe barrel safely housing the used needle can now be disposed of.

1. A hypodermic safety syringe assembly including a cylindrical barrel having a front end defining a first aperture and a rear end defining a second aperture, a closure member shaped to engage the barrel at or adjacent the front end thereof to seal the first aperture, and a piston assembly reciprocally movable within the barrel and including a retractor terminating in a piston formation engageable with the closure member to disengage the closure member from the front end of the barrel so that a hypodermic needle attached thereto can be withdrawn into the barrel after use, wherein the closure member includes a barrel engaging formation releasably engageable within a closure member engaging formation at the front end of the barrel, and a piston coupling formation for engaging a closure member coupling formation on the piston, with inter-engagement of the piston and closure member coupling formations being arranged to facilitate disengagement of the barrel engaging and closure member engaging formations from one another and to allow withdrawal of the piston assembly, closure member and hypodermic needle into the barrel.

2. The assembly of claim 1 wherein the closure member includes an inner skirt portion connected to an outer skirt portion by a hinge, with a displacement groove located between the inner and outer skirt portions.

3. The assembly of claim 2 wherein the barrel engaging formation includes a lip extending from the outer skirt portion, and the closure member engaging formation includes a lip receiving recess adjacent the front end of the barrel.

4. The assembly of claim 3 wherein the piston coupling formation includes at least one first projection and a corresponding projection receiving recess located radially outwardly of the first projection, and the closure member coupling formation includes a second projection and a corresponding recess located radially inwardly of the second projection, with the second projection being arranged to displace the first projection inwardly about the hinge during inter-engagement of the piston coupling formation and closure member coupling formation so as to urge the outer skirt formation, from which the first projection depends, inwardly so as to disengage the lip from the lip receiving recess.

5. The assembly of claim 4 wherein the first projection is a first annular lobe formation and the second projection is a second annular lobe formation.

6. The assembly of any one of the above claims including detaining means for detaining the used needle in a locked safe position within the barrel.
